(19) United States
(12) Patent Application Publication
(10) Pub. No.: US 2026/0272330 A1
(43) Pub. Date: Sep. 17, 2026

(54) Vorrichtung und System zur digitalen Überwachung und Unterstützung von Bewegungsabläufen

(71) Applicant: PANTHR HEALTHTECH GMBH, München (DE)

(72) Inventors: Alexander KRAUSE, München (DE); Thorsten DARGATZ, Hamburg (DE); Stefan TOPP, Hockenheim (DE)

(21) Appl. No.: 19/168,924

(22) PCT Filed: Mar. 25, 2024
(86) PCT No.: PCT/EP2024/057969

§ 371 Date: Feb. 11, 2026

(30) Foreign Application Priority Data
Mar. 27, 2023 (DE) 10 2023 107 586.1

(51) Int. Cl. A61B 5/11; A61B 5/00

(52) U.S. Cl. CPC A61B 5/1116; A61B 5/0022; A61B 5/0024; A61B 5/486; A61B 5/6805; A61B 5/7267; A61B 5/7405; A61B 5/7455

(57) ABSTRACT

A system with an intelligent garment and a corresponding external evaluation and communication system for digital monitoring and support of movement sequences is disclosed, wherein a digital capture and recording of body movements and a provision of a feedback to a wearer is performed in order to support the wearer in the movement sequences, and wherein the external evaluation and communication system is suitably connected to the intelligent garment for the purpose of data exchange, so that the external evaluation and communication system can carry out an evaluation of the actual data captured and transmitted by the intelligent garment. A determination of movement patterns of the wearer from the evaluated actual data and a generation of feedback signals is provided based thereon for a correction of the movement patterns of the wearer by means of the intelligent garment and can transmit these feedback signals to the intelligent garment.

### FIELD OF THE INVENTION

The present invention relates to an intelligent garment, a corresponding evaluation and communication system for digital monitoring and support of movement sequences and to a system with such an intelligent garment and such an evaluation and communication system.

### BACKGROUND OF THE INVENTION

In over 80 percent of cases, lack of movement, weak musculature and muscular imbalances are the cause for the continuously increasing number of patients, who suffer from subacute or chronic back pain. People who do not move enough and who sit for too long are particularly affected. According to a WHO study published in 2022, 44 percent of men and 40 percent of women do not move enough. According to a survey published in 2021, people with predominantly sedentary occupation (more than 50 percent of German citizens) additionally spend between 8.5 and 10.5 hours each day sitting down. Most of that time is spent in the office or in front of the television.

Due to the musculature, which is too weak, it is not possible to sit upright for a prolonged period of time. The result of this is a slouch with shoulders leaning forward. This leads to a shortened pectoral musculature and overstretched musculature in the upper back. As a result, neck tension and problems in the region of the cervical spine occur. At the same time, the muscles of the hip flexor tighten. Without countermeasures, the muscles shorten and harden in this region. The blood circulation thereof deteriorates and they no longer receive nutrients. The fascia also shorten and lose their elasticity. When standing or when walking, the hip flexion musculature has to then compensate for the shortened and tightened musculature, in that it acts on the back extensor musculature. The latter has to tighten in order to be able to counteract this. This creates excessive pressure on the joints, the vertebra and the intervertebral disks in the lumbar spine. This leads to incorrect posture, signs of wear and tear and pain. In this context, physicians call this non-specific back pain.

The best proven countermeasure in the case of non-specific back pain is a regular, sufficiently intensive muscle training, which is performed anatomically correctly and in an individually adapted manner, for example a regular strength training with one's own bodyweight, free weights (e.g., dumbbells) as well as resistances (e.g., cable pulleys, resistance bands). The disadvantage hereby is that approximately 80 percent of all people, who train without professional supervision, perform movements incorrectly. This reduces the training success because, for example, the wrong muscles are controlled. In addition, incorrect movement patterns take hold, which carry over into everyday behavior.

For example, physical therapists offer professional supervision, whereby the number of prescribed units per year is very limited and is not sufficient to sustainably and regularly strengthen the musculature. This is why relapsing (recurrent) subacute and chronic back pain is widespread. For time reasons, it is additionally not possible during physical therapy to either focus on muscular imbalances or to teach muscle-building exercises to the patients in such a way that they can continue to train safely on their own afterwards. The error rate when performing exercises during unsupervised training is more than 80 percent. Working with a personal trainer is a cost-intensive option. The success depends on the quality of the trainer. “Personal trainer” is not a protected professional group, anybody can call themselves that. There is thus the risk of ending up with a personal trainer with inadequate sports-related scientific and anatomical knowledge. In addition to the variable quality of the training support, the lack of objectification of the training performance and progress monitoring for a longer period of time are also disadvantages. Training at a fitness studio is more affordable. There, one has the option of working out on machines as well as with free weights. The quality of the training support depends on the quality of the studio thereby. Research shows that approximately 70 percent of all people, who train in a fitness studio, perform exercises anatomically incorrectly.

An EMS training has been offered for some time. EMS stands for Electrical Muscle Stimulation. People who train wear a special suit, which transmits electrical impulses to the muscles while performing mostly isometric (static) exercises, whereby the muscle building has been proven to be very effective but not particularly functional. The static exercises have little to do with everyday life and a correct movement performance is also not ensured here with poor support. In addition, the intensity control requires considerable experience. Research shows that over-training occurs frequently.

Training on special power machines, which guide the movements, whereby movement errors occur rarely, is also possible. However, only the superficial muscles are trained. However, for the everyday usability of the musculature, it is important to train the low-lying autochthonous musculature. Smart fitness equipment provides an improvement. However, this fitness equipment is tied to fixed locations (e.g., fitness studios) and is very cost-intensive. The feedback is unlimited and the training on the equipment leads to a guided movement, which is thus not suitable for everyday use. This neither trains the autochthonous musculature nor does it address the neuromuscular interaction.

Fitness apps are also used for training. Many apps ask for the fitness level and offer superficially adapted training programs. However, there is no feedback whatsoever with regard to the movement performance. Training control and progress monitoring is also possible only to some extent. The same is true for video-based fitness programs (e.g., on YouTube), whereby training support is not available here. Meanwhile, camera-based solutions by means of smartphones are also in use. Due to the limited field of view of the camera, a constant new positioning and repositioning of the smartphone is necessary. In addition, a correspondingly large distance from the person, who is to be trained, is necessary in order to be able to capture the entire body. But even if all of these conditions are met optimally, it is not always possible to capture all relevant points for a correct movement performance. In addition, this neither addresses the neuromuscular interaction between brain and working musculature, nor is a comprehensive evaluation of the performed exercises possible. The smartphone is furthermore often eliminated as visual feedback provider because for the optimal camara positioning it is mostly located outside the immediate field of view of the person, who is training.

It is thus desirable to have a digital monitoring and support of movement sequences at ones disposal, which can provide live feedback, like an actual personal trainer as well as overcomes or at least reduces the above disadvantages of the prior art.

### SUMMARY OF THE INVENTION

It is thus the object of the invention to provide a device for digital monitoring and support of movement sequences, which overcomes or at least reduces one or several disadvantages of the prior art, and which can in particular provide live feedback about the movement sequences.

The object is solved by means of an intelligent garment with an electronic network, which is integrated into the intelligent garment, for digital capture and recording of body movements and for provision of a feedback to a wearer of the intelligent garment, wherein the electronic network comprises at least
a plurality of sensor nodes comprising one or several sensors for digital capture of actual data from the wearer,
a storage unit for at least temporary storage of the actual data,
a sender unit for transmission of the temporarily stored actual data to an external evaluation and communication system, the transmission thereby preferably takes place wirelessly,
a receiver unit for reception of a feedback signal from the external evaluation and communication system for direct correction of a movement performance on the basis of the transmitted actual data and
a plurality of output units for provision of the feedback to the wearer on the basis of the received feedback signal from the external evaluation and communication system for correction of a movement performance by the wearer,
   wherein the above units are connected to one another in the electronic network and to a power supply of the electronic network by means of data and/or power connections, wherein the data and/or power connections are integrated into the intelligent garment.

Here, the term “a” or “an” does not refer to a certain number of the respective components referred to therewith but also explicitly includes the option that, in addition to the one component, the claimed subject matter can also comprise one or several further of these components. The term is to thus also be understood as “one or several”. If, in contrast, this refers to the number of the specified components, which are equal to 1, this is identified with “exactly one”.

Here, the term “intelligent garment” (or also referred to as iWear) refers to garments for covering the body, such as, for example, undergarments, socks, pants, T-shirts, sweatshirts, pullovers, jackets, shoes, gloves, hats, etc., which are equipped with electronic devices and functions, for example, sensor nodes, storage units, sender units, receiver unit, output units. It is thereby a special feature of this clothing that the electronic devices are not visible for the most part and that conductor tracks are generally woven into the textiles. The material of the intelligent garment can be any material, which is suitable for this purpose, the intelligent garment is thereby preferably tightly fitted to the body of the wearer. The wearer can also be referred to as user. The material can thus preferably be an elastic material. In contrast to intelligent garments, ordinary functional clothing only acts passively due to the properties of the used fabrics and materials, without comprising electronic devices and functions thereby. The term “intelligent garment” likewise does not comprise electronic devices, which can be worn on the body, such as so-called active trackers or smart watches, because these are worn separately from the garments and are not integrated into them.

The term “sensor node” thereby refers to a unit, which comprises one or several sensors for the determination of identical or different actual data for the body of the wearer, wherein the actual data determined by means of the respective sensor node is transmitted to the storage and/or sender unit via the electronic network. The plurality of sensor nodes is thereby suitably distributed over the intelligent garment, the sensor nodes preferably cover the intelligent garment in such a way that the movements of the wearer in the body parts, which the intelligent garment covers when being worn, can be monitored completely. The sensors of a sensor node can be, for example, acceleration sensors, gyroscopes, magnetometers, temperature sensors, moisture sensors, conductivity sensors, pressure sensors or other suitable sensors. The actual data is corresponding measuring data from the sensor nodes or the sensors of the sensor nodes, respectively, which is stored and transmitted as individual value or as average value of a series of measurements of a time interval as actual data. The respectively used format of the actual data can be suitably selected by the person of skill in the art or results from the type of the used components in the electronic network.

The units of the electronic network are thereby connected to one another by means of suitable data and/or power connections. Depending on the unit, which is to be connected, and on the provided supply of the unit, these connections can be realized wirelessly and/or in a wire-supported manner. In the case of wires, the latter preferably have a small cross section and a sheath, which is as flexible as possible. The cross section is preferably similar or smaller than the cross section of the individual fibers, from which the intelligent garment is produced. In the case of fixed material regions of the intelligent garment, for example leather sections of shoes, the wires can also be guided within the material. Alternatively, the wires can also be fastened to a surface of the intelligent garment, preferably the outer surface, by means of a suitable joining method, for example laminated, adhered or welded thereto. The wires for power and/or data connections (provided that no wireless connection is used), are integrated into the intelligent garment in order to ensure an improved wearing comfort for the wearer. “Integrated” means here that the cables or wires, respectively, of the connections are not guided separately from the clothing or cannot hang down from it, not even partially. The wearer can thus neither get stuck on the cables or wires, nor can they get stuck on objects in the surrounding area of the wearer. The power and/or data connections can, for example, be woven in or guided within the material of the garment or can be fastened, preferably laminated, thereto by means of a suitable joining method. The electronic network further comprises a power supply. The latter can be embodied in any suitable way. The power supply is preferably a rechargeable battery, preferably a flat button cell for reasons of wearing comfort. The intelligent garment and the electronic network are equipped completely for the wearer with the individual components, all required components are present in the electronic network. The user can thus use the intelligent garment immediately without modifications.

In the electronic network, the sender unit can thereby be designed as central transmission unit to the external evaluation and communication system. The term “external” hereby refers to the fact that the evaluation and communication system is not part of the intelligent garment or is not integrated into the intelligent garment, but is embodied separately from the intelligent garment. Due to a central sending unit, the number of components of the electronic network can be kept as small as possible, which lowers the complexity of the electronic network and the power consumption. In an alternative embodiment, however, the electronic network can also comprise a plurality of sender units, which simultaneously transmit the captured actual data to the external evaluation and communication system, which increases the data transmission rate to the external evaluation and communication system. The sender unit can hereby be any sender unit suitable for this purpose. Depending on the embodiment, the sender unit can transmit the data in different suitable formats to the corresponding receiving unit of the external evaluation and communication system. The sender unit preferably does this wirelessly, preferably by means of a Bluetooth connection and corresponding interfaces so as not to interfere with the movement sequences of the wearer and so as to ensure a continuous monitoring of the movement sequences.

The receiver unit has to be designed to be able to receive the feedback on the part of the external evaluation and communication system. The same as has already been described for the sender unit applies here. Sender and receiving unit can be formed, for example, as common sending and receiving unit. Suitable sender units, receiving units and sending and receiving units are known to the person of skill in the art. In the case of several sender units, which are arranged in a decentralized manner in the electronic network, the receiving unit can be formed separately therefrom as central receiving unit, because it is not known from the start, which body part the feedback to be output is to refer to and which output units are to convey the feedback to the wearer. In an embodiment with a single output unit in the electronic network, the receiver unit can be integrated into the output unit.

The output unit can hereby be any output unit, which is suitable to output a perceivable feedback to the wearer of the intelligent garment. For this purpose, the output unit can comprise a light source in order to output a visual feedback and/or can comprise actuating elements in order to output a tactile feedback and/or can comprise a loudspeaker in order to output an acoustic feedback. The feedback can thereby take place as short, long or modulated signal in a visual, acoustic or tactile manner.

The garment according to the invention thus differs, for example, from products, which do in fact comprise sensors for body parameters (IMU sensors) but which cannot output a feedback or a targeted feedback to the wearer. Such systems, which are low on feedback, cannot offer a comprehensive, individually adapted training planning or training control, as it is possible by means of the intelligent garment according to the invention. This movement control according to the invention can also not be achieved with the use of electromyography (EMG). The disadvantages of EMG lie in the accuracy of the captured data because only the muscle activity of large or surface muscles are used for the analysis there. However, muscle activity is only an indirect representative value for movement and is thus not suitable to provide a truly precise support of movement sequences.

The intelligent garment thus provides a device for digital monitoring and support of movement sequences, which can provide a timely feedback about the movement sequences. The intelligent garment in combination with the external evaluation and communication system provides for the evaluation of the biomechanical data, which would otherwise only be possible with the help of experts, and furthermore provides live feedback and can thus provide for the neuromuscular interaction in particular in response to a tactile feedback. With the intelligent garment according to the invention, it is not absolutely necessary to wear an entire suit and to manually attach sensors to the body of the wearer. The intelligent garment according to the invention is thus user friendly and requires neither a setup and calibration effort, which is to be carried out by experts, nor the presence of specially trained personnel.

In one embodiment, the plurality of sensor notes are arranged along strategic axes of the intelligent garment for surface monitoring of the wearer. The sections of the intelligent garment, which cover movable body parts, are referred to as strategic axes of the intelligent garment. In the case of a T-shirt, this would be, for example, the two arms and the back, because the arms and the back can be moved separately from one another. The strategic axis of the back thereby runs parallel to the spine, the strategic axes of the arms thereby run along upper arm and forearm. A strategic axis of the upper body can additionally run from the shoulder to the neck in order to cover the shoulder region. In the case of pants, the strategic axes would run from the pelvis along the upper leg and lower leg. In the case of gloves, the strategic axes would run along the phalanges and from the forearm via the wrist to the back of the hand. The person of skill in the art can further refine and supplement the strategic axes, depending on the garment. At least the strategic main axes, such as arms, legs and/or spine, are thus covered, the body of the wearer is thus monitored completely covering a large area. Movement sequences of the wearer can be comprehensively supported and monitored therewith. Several sensor nodes are thereby preferably arranged for each strategic axis.

In a further embodiment, the actual data comprises a sensor identification or sensor node identification. The actual data can thus be assigned to a certain sensor/sensor node and the movement pattern of individual body parts or limbs of the wearer can thus be recognized via the arrangement of the sensor nodes, which stretches across them, and the interaction of the different actual data from this arrangement at sensor nodes.

In a further embodiment, the sensor node comprises a three-axis acceleration sensor and/or a three-axis gyroscope and/or a magnetometer. The movement sequence can be captured completely and precisely by means of the actual data captured by means of these sensors, which is important for a high-quality support of movement sequences, which is based thereon.

In a further embodiment, one of the sender units is in each case arranged in the electronic network for each strategic axis. In the case of only one strategic axis on the intelligent garment, exactly one sender unit would be present only then.

However, the intelligent garment usually has several strategic axes (in the case of a T-shirt, for example, at least three strategic axes (along the spine, left arm, right arm, optionally additionally one axis from the right to the left shoulder, etc.). The data transmission rate to the external evaluation and communication system can thus be increased and the support of the movement sequences by means of a correspondingly quick feedback based on the faster data transmission can be improved by the output unit. The transmitted data can hereby be assigned immediately to the respective body parts of the wearer, for example by means of specific identification of the strategic axis for the corresponding sender unit in the transmitted signals, which further accelerates the feedback about the movement sequence.

In a further embodiment, the intelligent garment is at least partially made of a material, which ensures a positional accuracy of the sensor nodes on the body of the wearer when wearing the intelligent garment. In order to be able to correctly monitor the movement sequences over a longer period of time, the sensor node has to remain as much as possible at the same position (positional accuracy) relative to the body of the wearer. This positional accuracy can be improved by the selection of a suitable cutting pattern, preferably with a close-fitting cut of the intelligent garment and the selection of the material, which is suitable for this purpose, of the intelligent garment. The material preferably does not stretch or stretches only slightly with rising temperature and after moisture absorption as a result of sports activity or is pretensioned accordingly, respectively.

In a preferred embodiment, the material is an elastic textile thereby. The intelligent garment thus fits reliably tightly to the body of the wearer, at least in the regions of the garment, in which sensor nodes are arranged. The intelligent garment can hereby be made completely or partially of the elastic textile. In regions, in which no sensor node is arranged, the intelligent garment can be made of a different material. This different material should preferably allow for the weaving in of electrical connections, as in the case of the elastic textile. The elastic material ensures a tight and positionally accurate fit of the intelligent garment at least in the region of the sensor node on the body of the wearer even in response to sports-related heat-up of the body and absorbed sweat.

In a further embodiment, the sensor node is irreversibly connected to the material. The mechanical and electrical protection of the wearer against unwanted disengagement of the sensor node from the intelligent garment and thus a possible mechanical and/or electrical threat to the wearer due to detached sensor nodes with an undesired position relative to the body of the wearer is thus prevented. For example, the sensor node can thereby be interwoven with the material of the garment, adhered thereto, welded thereto or printed thereon.

In a further embodiment, the data and/or power connections are integrated, preferably interwoven, into the intelligent garment. The data and power connections are thus a natural part of the intelligent garment and not a component applied superficially, which may reduce the wearing comfort. Superficially applied data or power connections additionally have an increased risk of being damaged or even interrupted by the user, in particular by putting on and taking off the intelligent garment or a washing process for the intelligent garment.

In a further embodiment, the sensor nodes have a surface expansion along a surface of the intelligent garment, which is at least one order of magnitude larger than the thickness thereof perpendicular to the surface. The sensor nodes thus have a low center of gravity relative to the surface of the intelligent garment and thus also to the body surface of the wearer of the intelligent garment. Due to the shape of the sensor node, which is expanded on the surface, said sensor node exerts a correspondingly low pressure on the skin of the wearer because the weight of the sensor node is distributed to a larger area and can thus not be perceived or can only still barely be perceived by the wearer.

In a further embodiment, the power supply in the intelligent garment is arranged at a position, which, when the intelligent garment is worn, is located as close as possible to a center of gravity of the wearer when the wearer stands upright. The center of gravity of a person changes due to his/her body posture. In the case of an upright standing person, the center of gravity lies in the region of the abdomen or the hip, respectively. Provided that the intelligent garment does not cover hip or abdomen, the power supply is to be arranged in the intelligent garment so that it is located as close as possible to hip or abdomen when wearing the intelligent garment. This applies for pants of all types, outerwear, so-called bodysuits or overalls or other full body clothing. In the case of socks, hats, gloves or shoes, the power supply should, in contrast, be arranged at a different position, which does not make the wearing of these garments more difficult.

In a further embodiment, the sensor nodes and/or storage unit are designed to provide the captured actual data with time stamps, which are synchronized for the electronic network, for the time of capture of the actual data. Based on the time stamps, actual data relating to movement sequences of different sensor nodes, for example of different body parts, can be directly correlated with one another. The analysis of the performed movement sequence thus becomes significantly more precise, whereby an improved support of the movement sequence is made possible by means of feedback.

In a further embodiment, the electronic network is designed to delete the actual data from the storage unit after transmission to the external evaluation and communication system. Less storage volume is thus required and the storage of the electronic network can be embodied to be smaller. This also improves the required power consumption of the intelligent garment and the wearing comfort thereof.

In a further embodiment, the electronic network is designed to subject the captured actual data to a pre-processing prior to the transmission to the external evaluation and communication system. This pre-processing can be carried out, for example, by means of a data processing unit, which is either embodied as central additional component in the electronic network or which is integrated into a central transmission unit, respectively, or the processing unit is integrated into the respective sensor node in a decentralized manner. The processing unit can be embodied, for example, by a microprocessor, on which a corresponding processing program is installed and is run, whereby, for example, a noise is filtered out of the actual data, a normalization is carried out, or partial movements are recognized on the basis of a combination of movement data from one or several sensor nodes.

In a further embodiment, the output unit outputs a feedback in tactile form to the wearer, which feedback is designed for emulation of a touch from a trainer at its position, the feedback preferably takes place only after a repeated incorrect performance by the wearer has been determined. The incorrect performance comprises an incorrect positioning, an incorrect performance speed or an incorrect performance consistency. An incorrect positioning can further be subdivided into different aspects: an incorrect movement pattern—an incorrect dynamic posture—the asymmetry of a movement, in particular based on the sagittal plane, or an incorrect movement amplitude. Based on an individual exercise performance, an incorrect performance consistency describes an irregular movement speed, for example faltering or intense shaking. The performance speed can differ in the concentric and the eccentric phase, provided that this is specified explicitly. Based on several repetitions of an exercise, an incorrect performance consistency is a strong deviation of the movement patterns from one another. Here, the tactile perception serves the purpose of the passive perception of pressure, touch and vibrations as well as of the temperature because the output unit in the intelligent garment is located positionally accurately on or close to the skin of the wearer. The responsible sensory organ is the skin, namely the feel as well as heat and cold receptors thereof. The stimuli triggered by them are transmitted to the brain at high speed by the tactile nerve fibers via the spinal cord. The support of the movement sequence is thus experienced as a familiar correction, which improves the willingness of the wearer for the correction, whereby the support of the movement sequence is improved and intensified by the intelligent garment. The tactile feedback can be output, for example, via an actuator as output unit. This feedback preferably takes place only after repeated incorrect performance, so that random incorrect movements can be differentiated from systematic incorrect movements.

In a further embodiment, the output unit is a vibration motor or an EMS electrode. The desired tactile impressions can thus be generated reliably.

In a further embodiment, the plurality of output units are arranged along strategic axes of the intelligent garment and are provided to initiate a corrective movement of the wearer by means of the feedback. By means of the plurality of output units, a feedback is generated, preferably in tactile form, which triggers the effect of a neuromuscular interaction. The actuators can hereby also be positioned along the same anatomical axes as the sensor system for recording of the movement. Several output units for each strategic axis are preferably arranged for this purpose. The positions are determined in that a corrective movement is to be initiated along these axes by the tactile feedback. This neuromuscular interaction has the result for the wearer that the corresponding musculature can be controlled selectively or that a correction in the respective region becomes possible, respectively. This leads to a linkage of brain and muscle.

In a further embodiment, the sender unit has a Bluetooth or a WLAN interface. These interfaces are well suited for a reliable data transmission by means of Bluetooth or a WLAN connection and corresponding coupling to the external evaluation and communication system.

In a further embodiment, the intelligent garment is outerwear, preferably a T-shirt. By using the Tensegrity model (or in consideration of the myofascial lines), errors in movement sequences can be corrected not only in the upper body region but also in the region of the lower body by means of the garment according to the invention. This is so because, independently of the individual task of a muscle, the latter also influences functionally integrated connections, which encompass the entire body. Entire muscle groups are woven into the fiber orientation of the fascia and form myofascial connections, which can be tracked over the entire body. In these fiber groups, the muscles contract and transmit myofascial forces. As a result, upper body movements, which are performed anatomically correctly, also lead to movements of the lower body, which are performed anatomically correctly, and vice versa. An intelligent garment as T-shirt can thus significantly increase the safety during body exercises, even if the lower body half is not monitored. When additionally wearing an intelligent pair of pants as a further intelligent garment, for example a so-called sports leggings, the monitoring result can be improved even further or the neuromuscular interaction can also take place in regions of the lower body, respectively.

The invention furthermore relates to an evaluation and communication system for digital monitoring and support of movement sequences by means of data exchange with an intelligent garment according to the invention, wherein the evaluation and communication system is embodied separately from the intelligent garment as external evaluation and communication system, comprising
a data receiver for reception of captured actual data from the intelligent garment,
an evaluation unit for evaluation of the actual data, for determination of movement patterns of the wearer from the evaluated actual data and for generation of feedback signals based thereon for correction of the movement patterns of the wearer of the intelligent garment and
a transmission unit for transmission of the feedback signals to a receiver unit of the intelligent garment for subsequent provision of feedback to the wearer based on the determined movement patterns of the wearer.

The external evaluation and communication system can thereby be embodied as computer, server, cloud solution or smartphone or a combination thereof, wherein the cloud solution or the individual components, respectively, of the external evaluation and communication system are run on a suitable device in the surrounding area of the wearer of the intelligent garment. During the evaluation of the movement data, either the actual data from an individual sensor node (one position) or from a combination of several positions is used. In both cases, the position of the sensors on the body is to be considered and the limitations associated therewith, which are predetermined by the human anatomy—in which directions a sensor can move or how two or several positions (sensor nodes), respectively, can move relative to one another. For this purpose, the actual data can comprise a sensor identification, so that the transmitted actual data can be uniquely assigned to each individual sensor or sensor node, respectively. Any feedback signal can thereby consider previously determined normal position, fitness level/strength potential and mobility/flexibility of the wearer of the intelligent garment. The data receiver for reception of captured actual data can be any receiving unit, which is compatible with the sender unit of the intelligent garment. The transmission unit for transmission of the feedback signals can be any sending unit, which is compatible with the receiver unit of the intelligent garment.

The external evaluation and communication system according to the invention thus provides a device for digital monitoring and support of movement sequences, which determines the movement sequences and which can transmit a timely feedback signal about the movement sequences to the intelligent garment. The external evaluation and communication system in combination with the intelligent garment thus provides for the evaluation of the biomechanical data, which would otherwise only be possible with the help of experts and furthermore provides live feedback and thus provides for the neuromuscular interaction. The external evaluation and communication system according to the invention requires neither a setup and calibration effort, which is to be carried out by experts, nor the presence of specially trained personnel at the wearer of the intelligent garment according to the invention after a first use and is thus user friendly.

In one embodiment, the evaluation unit evaluates the actual data in real time and determines the movement patterns of the wearer. By means of the real time evaluation, the intelligent garment can, during the continuous movement, provide feedback relating to this to the wearer and optionally correction instructions, whereby the wearer can immediately adapt his/her movement sequence and can feel the changes directly, which significantly improves the learning effect.

In a further embodiment, the evaluation unit is designed to store the actual data as time series and to analyze it by applying fixed rules and/or by applying machine learning models. As data analysis node, the evaluation unit collects the pre-processed movement data transmitted by the intelligent garment, intermediately stores it as time series in an internal database and analyses it by applying fixed rules, such as threshold values, relative comparison and deviation from sensor values at different points and ML models in order to determine quantitative and qualitative values of the currently performed exercise. The derived feedback is output via the transmission unit during the exercise, for the simple movement and position correction, and is output after the exercise to the wearer of the intelligent garment as exercise evaluation. The evaluation of the actual data can thereby comprise a recognition of intervals, a recognition of performance speed via the temporal analysis of the recognized intervals—both of the interval itself and the sequence thereof, a recognition of time under tension (TuT) and stress—interval plus exercise type, a recognition of incorrect movements (position over time), a recognition of incorrect performances (statically), a monitoring of the lower body, which are then converted into training instructions and tactile and/or audiovisual feedback.

The recognition of intervals takes place by the consideration of the actual data over time and the sequence of extreme values and zeros when plotting the actual data as a function of time, for example with a neutral position as zero position. Depending on the exercise type, individual or a combination of different actual data time series (acceleration, XYZ(t), magnet XYZ(t), gyro XYZ(t), etc.) lend themselves hereby. Different sensor node positions may likewise need to be considered, so that the end of an exercise interval is recognized as a whole, because different body parts and sensor a node position connected thereto in each case arrive earlier or later at an extreme point or a zero due to the anatomy.

The recognition of the performance speed takes place via the temporal analysis of the previously recognized intervals—the rise of edges as well as the total length of an interval. The assignment of the concentric and of the eccentric phase thereby takes place via the combination of the exercise type and of the sign of the derivation of the amplitude.

A further temporal analysis on the basis of the intervals is the recognition of rhythm or uniformity, respectively. All intervals are hereby compared over the entire exercise sequence (movement sequence during the exercises). The absolute temporal deviations, relative to the interval start, between the consulted points (e.g., extreme points, zeros, turning points), which result over several intervals, allow drawing conclusions to the uniformity of the exercise performance.

A trend analysis of the temporal deviation from the uniformity evaluation allows drawing conclusions to a fatigue, which occurs, specifically when the temporal deviations increase with the number of performed intervals. A further indicator for fatigue is a flattening of the extreme points over time.

For evaluation of the muscular stress during an exercise and, conversely, in order to increase strength and stamina, the so-called time under tension (TuT) is considered. In the simplest case, the interval length of the respective exercise can be used hereby. A consideration as a whole takes place via the calculation of the area under a movement curve (actual data as a function of time). Occurring fatigue as well as a muscular strain can be recognized as the noise with the help of harmonic waves on the movement path and in a different/lower frequency range.

Based on the interval and asymmetry recognition, incorrect movements can also be recognized during the comparison (calculation of the difference) with reference movement curves. Different reference movement curves, sensor positions and sensor types are used, depending on the performed exercise.

In contrast to the incorrect movements, an analysis of static (without temporal dimension) orientation, gyroscope or magnetic data from the respective sensors of the sensor nodes takes place at several sensor node positions (or body parts) during the recognition of incorrect positions. The actual data from the respective sensors is compared hereby to one another in consideration of the human anatomy or biomechanical constraints. Due to the different orientation of the sensor nodes at different locations, different partial body axes (strategic axes) and the angles thereof can be determined relative to one another. A deviation of the relative axis position compared to a reference model allow drawing conclusions to an incorrect position.

Anatomically present incorrect positions at the wearer of the intelligent garment are considered during the analysis of incorrect movements and incorrect positions. It can happen thereby that deviations from the reference model are not considered to be an error. The determination of incorrect posture is determined via a calibration process, which is performed once at the beginning. During the calibration, the wearer has to assume previously determined reference positions. Orientation of the sensor node positions and the angles of the partial body axes (strategic axes) are determined in the meantime and are stored as the basic foundation.

If a direct monitoring by sensor nodes takes place only on the upper body, the lower body can also be monitored implicitly by applying the Tensegrity model. The fact that the human body is a closely linked biomechanical system, in the case of which each partial body movement has a direct impact on other partial bodies, makes it possible to draw conclusions to incorrect movements or positioning of the lower body. Corrections to the lower body can furthermore also be achieved via corrective movements on the upper body, and vice versa. This applies specifically in the case of the feedback while performing the exercise.

All discrete calculations and rules can also be replaced by a so-called Convolutional Neural Network (CNN). Prior to the start of an exercise, video instructions for the exercise to be performed are demonstrated to the user via the human-machine interface of the evaluation and communication system, for example the smartphone. The key instructions are narrated thereby and are emphasized with overlays in the video. They can additionally be supplemented by haptic signals, for example by addressing the region of the target musculature in combination with acoustic instructions.

The training planning, i.e., the determination of the exercise types, number of exercises, repetitions and intensity, takes place on the basis of an assessment, in the case of which data is manually entered by the wearer and/or is automatically determined. The information provided by the wearer is essentially age, gender, height, weight, lifestyle and preexisting conditions. Fitness level and muscular imbalances are to be calculated via the data, which is captured in an automated manner from the assessment. For this purpose, the wearer performs several predefined exercises. Values, such as duration, number of intervals and asymmetries are determined hereby, see above explanations. In addition, manual inputs by the wearer via standardized input fields are also considered. This includes, e.g., personal feelings or performance values, which cannot be measured in an automated manner by means of the device.

The exercises are classified in order to determine suitable exercises for an assessment result. The classification hereby corresponds to the criteria, which are queried and determined during the assessment. This creates a firm link between exercises and corresponding abilities and needs of the user. Several key parameters for the exercise performance are used during the evaluation of each induvial exercise. These key parameters describe the exercise qualitatively and quantitatively by uniformity and speed of the exercise performance, asymmetries, degree of fatigue and the percentage of determined incorrect movements. The entire training progress picks up the key parameters for the exercise performance for the exercise evaluation and subjects then to a temporal or trend analysis, respectively.

In a further embodiment, the evaluation unit is provided to perform a comparison of the actual data from sensor nodes, which, with respect to a body anatomy, are located at opposite positions or at positions, which relate to one another, of the intelligent garment. To identify asymmetries in the movement sequence of the wearer, a direct comparison of opposite sensor positions takes place—e.g., left shoulder and right shoulder, left arm and right arm, upper body in the front and at the back, etc.—or between positions with a certain relationship to one another, e.g., upper back to lower back. These comparisons represent an example for opposite positions based on the body anatomy, which is a symmetrical anatomy with respect to the spine for humans. Attention is thereby paid, for example, to a simultaneous movement, actual data with identical time stamp is thus compared (both sensor positions start and end at the same time) and both sensor positions move and rest at the same spatial positions, which leads to identical inertial-actual data, which is shifted only by a fixed value. This fixed value is predetermined by the human anatomy.

In a further embodiment, the data receiver and the transmission unit are installed by a smartphone and the evaluation unit by means of an application and are provided so as to run on the smartphone or in a cloud. The intelligent garment and the application on the smartphone (mobile application) can communicate hereby (exchange data and signals) via a wireless low-energy interface—preferably Bluetooth Low Energy (BLE). The mobile application may be in contact with a background system (a so-called backend) via an existing Internet connection. The smartphone forms a human-machine interface (HMI) between the wearer with the intelligent garment and the external evaluation and communication system, the mobile application acts as data analysis node and provides a control unit/gateway for the intelligent garment. In its function as HMI, the mobile application provides for the creation of user profiles, the setup of wearables, audio-visual accompaniment and the performance of assessment, training planning, training and result presentation. In addition, a gesture control is possible by means of the intelligent garment. Due to a manual tapping on a defined sensor node (for example on the shoulder or upper arm position), the user could interact with the system. A pre-configured control function could be, for example, a start-stop signal for the training. This so-called tapping for the control can take place on the intelligent garment independently of the evaluation and communication system directly by the wearer or other persons. The tapping is clearly recognizable in the data signal of the IMU of the sensor nodes.

In a further embodiment, the application is designed to output additional audio-visual feedback about the determined movement patterns to the wearer of the intelligent garment via the smartphone. The feedback is synchronized thereby and is adapted to the currently performed exercise of the wearer in order to trigger an appropriate movement or position correction in combination with the output unit of the intelligent garment with the wearer (user). On the basis of the recognized incorrect movements, incorrect positions and temporal deviations (uneven, too quick, too slow), an additional audio-visual feedback is provided to the user, for example via the smartphone. This is limited to short instructions, which are easy to understand, which trigger a corrective movement with the wearer. Attention is paid to the fact that the feedback is not provided in the case of each insignificant error and also not too frequently (max. 1-3 times) during an exercise, according to the user preferences, analogously to the approach of a human trainer. The audio-visual feedback is provided in combination, synchronized, with a haptic feedback.

In a further embodiment, the application is provided to
query a status of the intelligent garment and/or
to transmit parameters and/or data necessary for operation of the intelligent garment and/or
to start or to end a training operation with the intelligent garment.
   As control unit/gateway for the intelligent garment, the application on the smartphone (mobile application) can provide the access to the internal status of the wearable—e.g., battery charge level, connection quality, operational readiness. New software revisions and other parameters/data necessary for the operation can furthermore be transmitted or transferred to the intelligent garment via the mobile application. When starting or ending the training operation, the mobile application can start or stop the recording and transmission of movement data into/from the intelligent garment. Temporally adapted to the audio-visual feedback via the smartphone, the mobile application can also transmit commands as necessary parameters/data to the intelligent garment in order to activate, for example, actuators at the matching positions for a tactile feedback to the wearer.

In a further embodiment, the external evaluation and communication system comprises a background system, to which the evaluation unit is connected via a suitable data connection, wherein the background system is designed to provide suitable business logic, security and other network services, services for machine learning (ML) as well as for storing and maintaining data for the evaluation unit and/or the intelligent garment. The background system (a so-called backend) can comprise partial applications for the corresponding application logic thereby and can implement security and other network services, services for machine leaning (ML), storages, interface management and orchestration in order to implement the following applications:
aggregation and long-term storage of the time series data from the individual training units.
Long-term data analysis for evaluation of training progress, trends, predictions and anomaly recognition.
Provision of interfaces for the secure granting of access to training and assessment data by authorized third parties—e.g., therapists or health service providers.
User profile management, authentication and authorization training and distribution of models for the movement capture and training evaluation at mobile application and intelligent garment
distribution of new firmware versions to the intelligent garment.

A permanent connection between evaluation unit and background system is not absolutely necessary hereby in order to fulfill the main purpose of the invention, namely the digital monitoring and support of movement sequences.

The invention furthermore relates to a system for digital monitoring and support of movement sequences comprising an intelligent garment according to the invention and an evaluation and communication system according to the invention, which is embodied separately from the intelligent garment as an external evaluation and communication system for the intelligent garment, wherein a digital capture and recording of body movements and a provision of a feedback to a wearer is performed by means of the intelligent garment in order to support the wearer in the movement sequences, and wherein the external evaluation and communication system is suitably connected to the intelligent garment for the purpose of data exchange, so that the external evaluation and communication system can carry out an evaluation of the actual data captured and transmitted by the intelligent garment, a determination of movement patterns of the wearer from the evaluated actual data and a generation of feedback signals based thereon for a correction of the movement patterns of the wearer by means of the intelligent garment and can transmit these feedback signals to the intelligent garment.

The system according to the invention thus provides a device for digital monitoring and support of movement sequences, which determines movement sequences and which can transmit a timely feedback signal about the movement sequences to the intelligent garment. By means of the external evaluation and communication system according to the invention in combination with the intelligent garment according to the invention, the system thus provides for the evaluation of the biomechanical data, which would otherwise only be possible with the help of experts and furthermore provides live feedback and thus provides for the neuromuscular interaction. The system according to the invention does not require a presence of specially trained personnel at the wearer of the intelligent garment according to the invention and is thus user friendly.

The above-described embodiments can be combined arbitrarily with one another by the person of skill in the art as part of the teaching according to the invention, also deviating from the claim dependencies.

### BRIEF DESCRIPTION OF THE DRAWINGS

FIG. 1: shows an intelligent garment according to the invention as T-shirt in the front view;

FIG. 2: shows a schematic illustration of a sensor node with several sensors (a) in top view and (b) in side view;

FIG. 3: shows a schematic illustration of a system according to the invention of intelligent garment according to the invention and external evaluation and communication system according to the invention; and

FIG. 4: shows a schematic illustration of the (a) actual data from different sensor nodes during the same exercise, and of the (b) actual data from an acceleration sensor as a function of time of a movement exercise with two successive performances.

### DETAILED DESCRIPTION OF THE DRAWINGS

The embodiments shown here only represent examples for the present invention and must thus not be understood in a limiting manner. Alternative embodiments considered by the person of skill in the art are likewise captured by the scope of protection of the present invention.

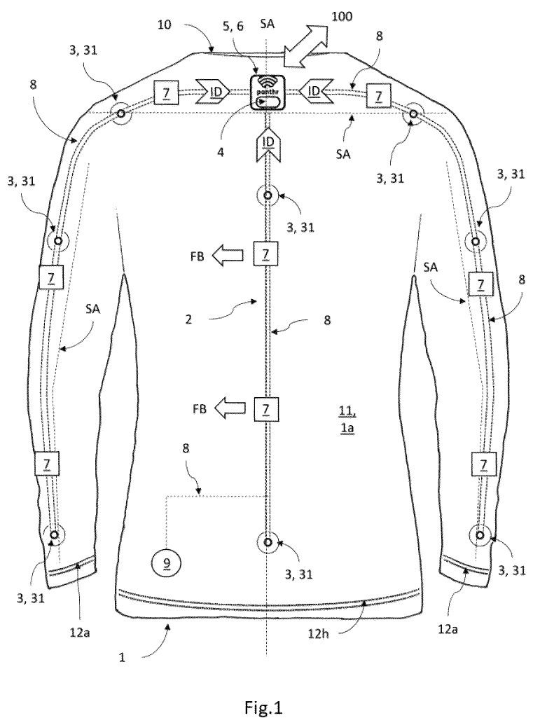

FIG. 1 shows an intelligent garment 1 according to the invention as long-sleeved T-shirt in the front view. The principle shown here can also be transferred to other outerwear. The T-shirt 1 comprises an integrated electronic network 2 for digital capture and recording of body movements and for provision of feedback FB to a wearer of the T-shirt 1. Here, the electronic network 2 comprises a plurality of sensor nodes 3, a storage unit 4, a sending unit 5, a receiver unit 6 and a plurality of output units 7. The T-shirt 1 is made completely of an elastic textile material 11 here, which ensures a positional accuracy of the sensor nodes 3 on the body of the wearer when wearing the intelligent garment 1. At least the sensor nodes 3 and the output units 7 are irreversibly connected to the textile material 11 here. The sensor nodes 3 thereby serve the purpose of capturing actual data ID from the wearer, which comprises a sensor identification or sensor node identification. Here, the sensor nodes 3 are arranged along strategic axes SA of the intelligent garment 1 for surface monitoring of the wearer. For illustration purposes, four different strategic axes SA (along the left arm, along the right arm, along the spine and from the left to the right shoulder) are illustrated here. Depending on the design of the intelligent garment 1, more or fewer strategic axes SA can also be defined and can be displayed accordingly in the electrical network 2 with corresponding connections and components. The storage unit 4 is provided for temporary storage of the actual data ID and is integrated in this case in a common component with sender unit 5 and receiver unit 6 between the shoulder sections just below the neckline 10. The sensor nodes 3 and/or the storage unit 4 can be designed thereby to provide the captured actual data ID with time stamps, which are synchronized for the electronic network 2, for the time of capture of the actual data ID. In addition, the electronic network 2 can be designed to delete the actual data ID from the storage unit 4 after transmission to the external evaluation and communication system 100. Here, the sender unit 5 is provided for a wireless transmission of the temporarily stored actual data ID, for example via a Bluetooth or a WLAN interface, to the external evaluation and communication system 100 (displayed in a symbolized manner). The electronic network 2 can thereby be designed to subject the captured actual data ID to a pre-processing prior to the transmission to the external evaluation and communication system 100, for example in the common component of storage unit 4, sender unit 5 and receiving unit 6, which, for this purpose, can comprise a processor and a corresponding pre-processing program, which is run on the processor. In other embodiments, one of the sender units 5 for each strategic axis SA can in each case also be arranged in the electronic network 2. The receiver unit 6 is provided for reception of a feedback signal FBS from the external evaluation and communication system 100 on the basis of the transmitted actual data ID. Here, the plurality of output units 7 are arranged along the strategic axes SA of the T-shirt 1 and are provided to initiate a corrective movement of the wearer by means of the feedback FB. A feedback FB can hereby take place only along one of the strategic axes SA, while the output units 7 along the other strategic axes SA do not provide any kind of feedback FB to the wearer, for example because the remaining movement percentages of the wearer are correct and because there is no need for correction there. In the example shown here, feedback FB is sent only by the output units 7, which are arranged along the spine (strategic axis) of the wearer, which is to correct a slouching back posture, for example. During the slouching back posture, the posture of the arms and of the shoulders relative to one another can be correct, for example, which is why the output units 7 at that location do not send out a feedback FB. The output units 7 can output this feedback FB, which is designed for emulation of a touch from a trainer at its position, to the wearer, for example in tactile form, the feedback FB is preferably provided only after a determined repeated incorrect performance by the wearer. Such an output unit 7 can be embodied as vibration motor or EMS electrode. The above units 3, 4, 5, 6, 7 are connected to one another in the electronic network 2 by means of data and/or power connections 8 and to a power supply 9 of the electronic network, wherein the data and/or power connections 8 are woven into the intelligent garment 1. The power supply 9 is hereby arranged at that position, which is located in the region of the hip of the wearer when wearing the T-shirt 1. When wearing the intelligent garment 1, the power supply 9 is thus located as close as possible to a center of gravity of the wearer when the wearer stands upright. In the case of the T-shirt 1, the two arm cuffs 12a and the waistband 12h are illustrated by a double line here. Neither the arm cuffs 12a nor the waistband 12h are part of the electronic network 2, but part of the T-shirt material per se.

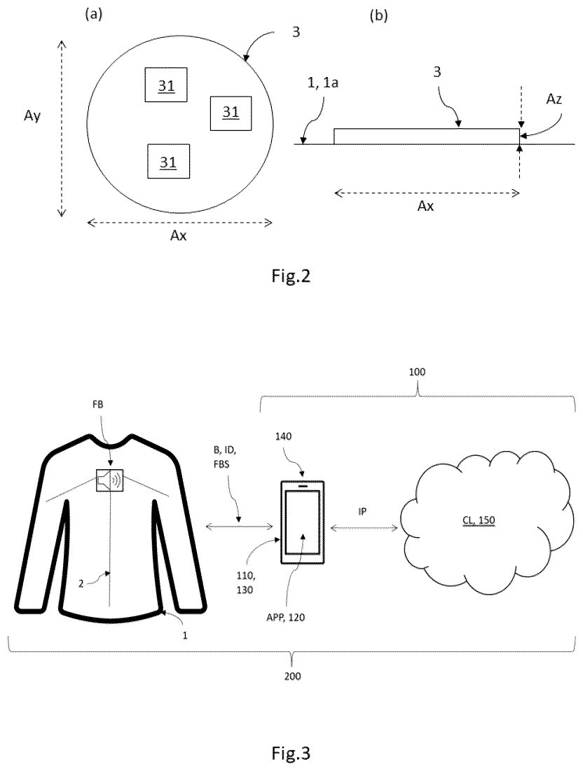

FIG. 2 shows a schematic illustration of a sensor node 3 with several sensors 31 (a) in top view and (b) in side view. In the embodiment shown here, the sensor node 3 has a surface expansion Ax, Ay along a surface 1a of the intelligent garment 1, which is at least one order of magnitude larger than the thickness Az thereof perpendicular to the surface 1a. The sensors 31 of the respective sensor node 3 can thereby be different sensors 31 with different measuring tasks. For example, the sensor node 3 can have a three-axis acceleration sensor 31, a three-axis gyroscope 31 and a magnetometer 31.

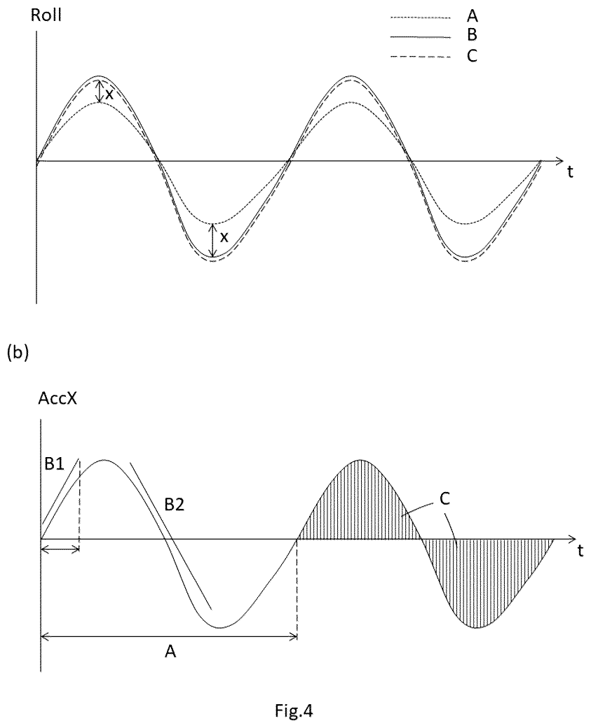

FIG. 3 shows a schematic illustration of a system 200 according to the invention of an intelligent garment 1 according to the invention and external evaluation and communication system 100 according to the invention, wherein a digital capture and recording of body movements and a provision of a feedback FB to a wearer is performed by means of the intelligent garment 1 in order to support the wearer in the movement sequences. For the details relating to the intelligent garment 1 according to the invention, reference is made to FIG. 1. The external evaluation and communication system 100 for digital monitoring and support of movement sequences is connected to the intelligent garment 1 here via a Bluetooth connection B with corresponding interfaces on both sides for the purpose of data exchange. The external evaluation and communication system 100 further comprises a data receiver 110 for reception of captured actual data ID from the intelligent garment 1, which data is transmitted via the Bluetooth connection B from the sender unit 5 of the intelligent garment 1 to the data receiver 110, an evaluation unit 120 for evaluation of the actual data ID, for determination of movement patterns of the wearer from the evaluated actual data ID and for generation of feedback signals FBS, which are based thereon, for correction of the movement patterns of the wearer of the intelligent garment 1, and a transmission unit 130 for transmission of the feedback signals FBS via the same Bluetooth connection B to a receiver unit 6 of the intelligent garment 1 for subsequent provision of feedback to the wearer based on the determined movement patterns of the wearer. The evaluation unit 120 evaluates the actual data ID in real time hereby and determines the movement patterns of the wearer. For this purpose, the evaluation unit 120 is thereby designed to store the actual data ID as time series and to analyze it by applying fixed rules and/or by applying machine learning models. It also carries out a comparison of the actual data ID of sensor nodes 3 hereby, which, with respect to a body anatomy, is located on opposite positions of the intelligent garment 1, in order to determine asymmetries in the movement sequences. In this embodiment, the data receiver 110 and the transmission unit 120 is installed by a smartphone 140 and the evaluation unit 120 by means of an application APP and is provided so as to run on the smartphone 140 or in a cloud CL. The application APP is further designed to output additional audio-visual feedback about the determined movement patterns to the wearer of the intelligent garment 1 via the smartphone 140. The application APP is further provided to query a status of the intelligent garment 1, to transmit parameters and/or data necessary for operation of the intelligent garment 1 and to start or to end a training operation with the intelligent garment 1. In this embodiment, the external evaluation and communication system 100 comprises a background system 150 (see bracket “100”), to which the evaluation unit 120 is connected via a suitable data connection, wherein the background system 150 is designed to provide suitable business logic, security and other network services, services for machine learning (ML) as well as for storing and maintaining data for the evaluation unit 120 and/or the intelligent garment 1.

FIG. 4 shows a schematic illustration of the (a) actual data with respect to the rotation R of different sensor nodes 3 during the same exercise, and of the (b) actual data “AccX” of an acceleration sensor 31 as a function of time of a movement exercise with two successive performances. During the evaluation/analysis of the movement data, either the actual data ID from an individual sensor node 3 (one sensor position) or from a combination of several positions is used. In both cases, the position of the sensor nodes 3 on the body is to be considered and the limitations associated therewith, which are predetermined by the human anatomy/biomechanical constraints—in which directions a sensor node 3 can move in a translatory and rotatory manner or how two or several positions of different sensor nodes 3, respectively, can move relative to one another. The following logics result from this for the recognition and evaluation of different aspects. When recognizing asymmetries, a direct comparison of opposite sensor positions takes pace (see FIG. 4a)—e.g., left and right, top and bottom or in the front and at the back. Attention is paid thereby to a simultaneous movement—both sensor positions start and end at the same time—and both sensor positions move and rest at the same spatial positions, which leads to identical inertial data (incline, rotation R, direction, acceleration XYZ) (see FIG. 4a—pos. B & C), which can be shifted by a fixed value (see FIG. 4a-x) . This fixed value is predetermined by the human anatomy. Compared to curve B and C, curve A does not display any asymmetry but a symmetrical movement. The shift x is anatomical but is not an asymmetry. Anatomical shifts can be differentiated from actual asymmetries, in that a comparison with the corresponding actual data from the calibration process is carried out.

The recognition of intervals takes place by the consideration of the actual data ID over time t and the sequence of extreme values and zeros when plotting the actual data ID as a function of time t, see FIG. 4b. Here, the exercise starts with a strong first initial acceleration B1, two following plateaus with a small local minimum, a strong reverse second acceleration B2 with zero crossing and return to the initial value. The same pattern repeats itself in response to the repetition of the first exercise performance. Due to the characteristic progression of the actual data over time, end and beginning of an exercise interval A can be recognized as a whole. The recognition of the performance speed takes place via the temporal analysis of the previously recognized intervals—the rise of edges B1, B2 as well as the total length A of an interval. The subdivision of the speed to the concentric and the eccentric phase takes place by the sign change (turning point) of the derivation/edge rise in combination with the knowledge of the exercise type. A further temporal analysis on the basis of the intervals is the recognition of rhythms or uniformity, respectively, of the movement amplitude and speed of the individual phases. All intervals are hereby compared over the entire exercise sequence (movement sequence during the exercises). The absolute temporal deviations, relative to the interval start, between the consulted points (e.g., extreme points, zeros, turning points), which result over several intervals, allow drawing conclusions to the uniformity of the exercise performance. A trend analysis of the temporal deviation from the uniformity evaluation allows drawing conclusions to a fatigue, which occurs, specifically when the temporal deviations increase with the number of performed intervals. A further indicator for fatigue is a flattening of the extreme points over time. For evaluation of the muscular stress during an exercise and, conversely, in order to increase strength and stamina, the so-called time under tension (TuT) is considered. In the simplest case, the interval length of the respective exercise can be used hereby. A consideration as a whole takes place via the calculation of the area under a movement curve (shaded area C of the actual data as a function of time). Occurring fatigue as well as a muscular strain can be recognized as the noise over time with the help of harmonic waves on the actual data history and with a different/lower frequency. Based on the interval recognition, incorrect movements can also be recognized during the comparison (calculation of the difference) with reference movement curves. Different reference movement curves, sensor positions and sensor types are used, depending on the performed exercise.

### List of Reference Numerals

1 intelligent garment according to the invention
1a surface of the intelligent garment
11 material of the intelligent garment
2 electronic network
3 sensor node
31 sensor(s) of the sensor node
4 storage unit
5 sender unit
6 receiver unit
7 output unit
8 data and power connections
9 power supply
10 neckline
12a arm cuff
12h waistband
100 external evaluation and communication system according to the invention
110 data receiver of the external evaluation and communication system
120 evaluation unit of the external evaluation and communication system
130 transmission unit of the external evaluation and communication system
140 smartphone
150 background system
200 overall system according to the invention
A length of an exercise interval
AccX actual data from an acceleration sensor
APP application installed and run on a smartphone
Ax, Ay surface expansion of the sensor node
Az thickness of the sensor node
B Bluetooth connection
B1 first acceleration phase
B2 second acceleration phase
C area under the curve of the performed exercise
CL cloud
FB feedback to the wearer
FBS feedback signal from the external evaluation and communication system
ID actual data from the wearer
IP Internet connection
SA strategic axes of the intelligent garment
R actual data from a sensor node with respect to t rotation
t time

1. An intelligent garment with an electronic network, which is integrated into the intelligent garment, for digital capture and recording of body movements and for provision of a feedback to a wearer of the intelligent garment, wherein the electronic network comprises at least
a plurality of sensor nodes comprising one or several sensors for digital capture of actual data from the wearer,
a storage unit for at least temporary storage of the actual data,
a sender unit for transmission of the temporarily stored actual data to an external evaluation and communication system, the transmission thereby preferably takes place wirelessly,
a receiver unit for reception of a feedback signal from the external evaluation and communication system for direct correction of a movement performance by the wearer on the basis of the transmitted actual data and
a plurality of output units for provision of the feedback to the wearer on the basis of the received feedback signal from the external evaluation and communication system for correction of a movement performance by the wearer,
wherein the above units are connected to one another in the electronic network and to a power supply of the electronic network by means of data and/or power connections, wherein the data and/or power connections are integrated into the intelligent garment.

2. The intelligent garment according to claim 1,
wherein
the one plurality of sensor notes are arranged along strategic axes of the intelligent garment for surface monitoring of the wearer, several sensor nodes are preferably arranged for each strategic axis.

3. The intelligent garment according to claim 2,
wherein
one of the sender units is in each case arranged in the electronic network for each strategic axis.

4. The intelligent garment according to claim 1,
wherein
the sensor node comprises a three-axis acceleration sensor and/or a three-axis gyroscope and/or a magnetometer.

5. The intelligent garment according to claim 1,
wherein
the actual data comprises a sensor identification or sensor node identification.

6. The intelligent garment according to claim 1,
wherein
the intelligent garment is at least partially made of a material, which ensures a positional accuracy of the sensor nodes on the body of the wearer when wearing the intelligent garment, the material is preferably an elastic textile.

7. The intelligent garment according to claim 6,
wherein
the sensor node is irreversibly connected to the material.

8. The intelligent garment according to claim 1,
wherein
the data and/or power connections are incorporated, preferably woven into, the intelligent garment.

9. The intelligent garment according to claim 1,
wherein
the sensor nodes have a surface expansion along a surface of the intelligent garment, which is at least one order of magnitude larger than the thickness thereof perpendicular to the surface.

10. The intelligent garment according to claim 1,
wherein
the power supply in the intelligent garment is arranged at a position, which, when the intelligent garment is worn, is located as close as possible to a center of gravity of the wearer when the wearer stands upright.

11. The intelligent garment according to claim 1,
wherein
the sensor nodes and/or storage unit are designed to provide the captured actual data with time stamps, which are synchronized for the electronic network, for the time of capture of the actual data.

12. The intelligent garment according to claim 1,
wherein
the electronic network is designed to delete the actual data from the storage unit after transmission to the external evaluation and communication system.

13. The intelligent garment according to claim 1,
wherein
the electronic network is designed to subject the captured actual data to a pre-processing prior to the transmission to the external evaluation and communication system.

14. The intelligent garment according to claim 1,
wherein
the output unit outputs a feedback in tactile form to the wearer, which feedback is designed for emulation of a touch from a trainer at its position, the feedback preferably takes place only after a repeated incorrect performance by the wearer has been determined.

15. The intelligent garment according to claim 14,
wherein
the output unit is a vibration motor or an EMS electrode.

16. The intelligent garment according to claim 1,
wherein
the plurality of output units are arranged along strategic axes of the intelligent garment and are provided to initiate a correction of the movement performance of the wearer by means of the feedback.

17. The intelligent garment according to claim 1,
wherein
the sender unit has a Bluetooth or a WLAN interface.

18. The intelligent garment according to claim 1,
wherein
the intelligent garment is outerwear, preferably a T-shirt.

19. An evaluation and communication system for digital monitoring and support of movement sequences by means of data exchange with an intelligent garment according to claim 1, wherein the evaluation and communication system is embodied separately from the intelligent garment as external evaluation and communication system, comprising
a data receiver for reception of captured actual data from the intelligent garment,
an evaluation unit for evaluation of the actual data, for determination of movement patterns of the wearer from the evaluated actual data and for generation of feedback signals based thereon for correction of the movement patterns of the wearer of the intelligent garment and
a transmission unit for transmission of the feedback signals to a receiver unit of the intelligent garment for subsequent provision of feedback to the wearer based on the determined movement patterns of the wearer.

20. The evaluation and communication system according to claim 19,
wherein
the evaluation unit evaluates the actual data in real time and determines the movement patterns of the wearer.

21. The evaluation and communication system according to claim 19,
wherein
the evaluation unit is designed to store the actual data as time series and to analyze it by applying fixed rules and/or by applying machine learning models.

22. The evaluation and communication system according to claim 19,
wherein
the evaluation unit is provided to perform a comparison of the actual data from sensor nodes, which, with respect to a body anatomy, are located at opposite positions or at positions, which relate to one another, of the intelligent garment.

23. The evaluation and communication system according to claim 19,
wherein
the data receiver and the transmission unit are installed by a smartphone and the evaluation unit by means of an application and are provided so as to run on the smartphone or in a cloud.

24. The evaluation and communication system according to claim 23,
wherein
the application is designed to output additional audio-visual feedback about the determined movement patterns to the wearer of the intelligent garment via the smartphone.

25. The evaluation and communication system according to claim 23,
wherein
the application provided to
query a status of the intelligent garment and/or
to transmit parameters and/or data necessary for operation of the intelligent garment and/or
to start or to end a training operation with the intelligent garment.

26. The evaluation and communication system according to claim 19,
wherein
the external evaluation and communication system comprises a background system, to which the evaluation unit is connected via a suitable data connection, wherein the background system is designed to provide suitable business logic, security and other network services, services for machine learning as well as for storing and maintaining data for the evaluation unit and/or the intelligent garment.

27. A system for digital monitoring and support of movement sequences comprising an intelligent garment and an evaluation and communication system according to claim 19, which is embodied separately from the intelligent garment as an external evaluation and communication system for the intelligent garment, wherein a digital capture and recording of body movements and a provision of a feedback to a wearer is performed by means of the intelligent garment in order to support the wearer in the movement sequences, and wherein the external evaluation and communication system is suitably connected to the intelligent garment for the purpose of data exchange, so that the external evaluation and communication system can carry out an evaluation of the actual data captured and transmitted by the intelligent garment, a determination of movement patterns of the wearer from the evaluated actual data and a generation of feedback signals based thereon for a correction of the movement patterns of the wearer by means of the intelligent garment and can transmit these feedback signals to the intelligent garment.
